(19) United States
(12) Patent Application Publication
(10) Pub. No.: US 2004/0267357 A1
(43) Pub. Date: Dec. 30, 2004

(54) Cardiac valve modification method and device

(76) Inventors: Jeffrey W. Allen, Santa Rosa, CA (US); David S. Brin, Topsfield, MA (US); Chris M. Coppin, Carlsbad, CA (US); Nasser Rafiee, Andover, MA (US); Theodore C. Lamson, Pleasanton, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/833,448

(22) Filed: Apr. 28, 2004

Related U.S. Application Data
(60) Provisional application No. 60/466,940, filed on Apr. 30, 2003.

(51) Int. Cl. A61F 2/24

(52) U.S. Cl. 623/2.11; 623/2.36

(57) ABSTRACT

The cardiac valve modification device of the present invention provides a catheter; an injection assembly disposed on the catheter, the injection assembly having lobes, and a neck disposed between the lobes; and at least one injector operably disposed at the neck. The injector can be an injection barb, made of a biodegradable material including a therapeutic agent, and deposited in the valve annulus, or an injection needle, which delivers a therapeutic agent to the valve annulus. The therapeutic agent can be a pro-fibrotic growth factor or a bulking agent. The cardiac valve modificaton method comprises inserting an injection catheter to the valve annulus; and injecting a therapeutic agent into the valve annulus with the injection catheter. The procedure can be repeated to modify the cardiac valve in a series of steps.

### RELATED APPLICATION

This application claims priority to U.S. Provisional Application No. 60/466,940, “Cardiac Valve Modification Method and Device” to Jeffrey W. Allen et al., filed Apr. 30, 2003, the entirety of which is incorporated by reference.

### TECHNICAL FIELD

The technical field of this disclosure is medical devices and methods for treating cardiac valves, particularly, a method and device for modifying cardiac valves.

### BACKGROUND OF THE INVENTION

Human heart valves, such as the aortic and mitral valves are sometimes damaged by diseases or by aging which cause problems with the proper function of the leaflets and/or the sub-valvular apparatus attached to the leaflets. These valves consist of a valve annulus of fibrous tissue to which the flexible leaflets are attached. Often, degenerative disease causes the valve annulus to dilate to the point where the leaflets attached to it cannot fully close and allow valve leakage. This inability to close completely, a condition called valve insufficiency or incompetence, eventually requires surgical correction either by valve repair procedures or by valve replacement. In common practice, both repair and replacement require open-heart surgery with its attendant risks, expense, and extended recovery time.

Catheter based valve repair systems to modify the valve annulus using heat and radio frequency (RF) energized electrode catheters to shape the valve annulus or injected material are known in the art. The catheter based systems access the heart valve through a small incision in the groin or neck, which avoids opening the chest as in open-heart surgery. A heating device on the distal end of the catheter is applied to or inserted below the surface of the valve annulus and heat is applied to shrink and tighten the collagenous tissue of the valve annulus.

Use of RF energized electrode catheters has significant disadvantages. For example, often when using such a catheter to form a cardiac lesion, the cardiac tissue becomes charred from the RF energized heating of the tissue, blood near the cardiac tissue undergoing treatment becomes coagulated, and the cardiac tissue undergoes separation and/or popping.

U.S. Pat. No. 6,267,781 to Tu discloses an ablation device for treating tissues, valvular annulus, valvular organ structure, or atherosclerosis of a patient, the ablation device including a flexible elongate tubular shaft having a deployable spiral wire electrode at its distal end adapted to contact/penetrate the tissue to be treated and to apply high frequency energy to the tissue for therapeutic purposes.

U.S. Pat. No. 6,355,030 to Aldrich et al. discloses methods and devices for improving valve function in a heart, including a thermal heating device as part of a thermal heating member fixed to an elongate member, the thermal heating device being inserted into working space proximate the valve to be treated and being used to selectively contract the collagen fibers of the valve structure treated so as to improve the performance and functioning of the valve.

U.S. Pat. No. 6,485,489 to Teirstein et al. discloses a delivery system and methods for repairing an annular organ structure comprising injecting a heat shapeable biomaterial formulated for in vivo administration by injection via a delivery system at a site of the valvular annulus defect; and applying heat sufficient to shape and immobilize the biomaterial at about the annulus defect, and optionally to shape tissue surrounding the annulus defect.

It would be desirable to have a cardiac valve modification method and device that would overcome the above disadvantages.

### SUMMARY OF THE INVENTION

One aspect of the present invention provides a cardiac valve modification device and method to change cardiac valve function by changing the flexibility and geometry of the valve annulus.

Another aspect of the present invention provides a cardiac valve modification device and method to inject a therapeutic agent into a valve annulus.

Another aspect of the present invention provides a cardiac valve modification device to locate an injection assembly at the valve annulus.

Another aspect of the present invention provides a cardiac valve modification method to gradually change cardiac valve function with a series of valve modifications.

The foregoing and other features and advantages of the invention will become further apparent from the following detailed description of the presently preferred embodiments, read in conjunction with the accompanying drawings.

The detailed description and drawings are merely illustrative of the invention, rather than limiting the scope of the invention being defined by the appended claims and equivalents thereof.

### BRIEF DESCRIPTION OF THE DRAWINGS

FIGS. 1-4C show an injection catheter for cardiac valve modification made in accordance with the present invention.

FIGS. 5 & 6 show another injection catheter for cardiac valve modification made in accordance with the present invention.

FIG. 7 shows a flowchart for a method of cardiac valve modification in accordance with the present invention.

FIG. 8 shows a flowchart for another embodiment of a method of cardiac valve modification in accordance with the present invention

### DETAILED DESCRIPTION

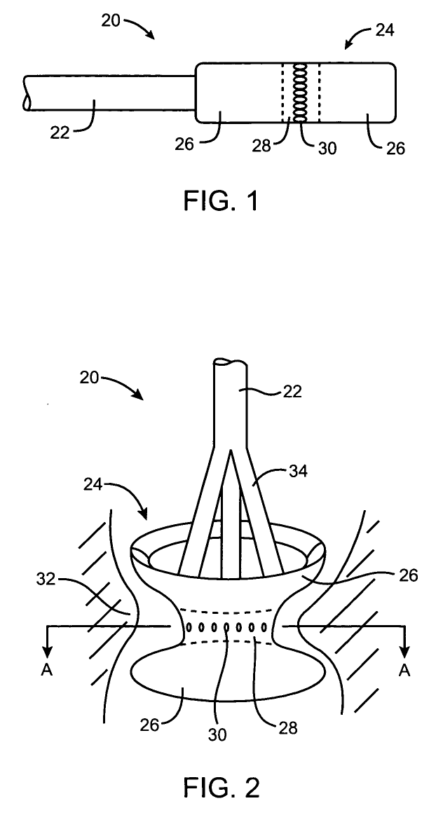

FIGS. 1-4 show an injection catheter for cardiac valve modification. The injection catheter locates injectors in a cardiac valve annulus to dispense a therapeutic agent. The therapeutic agent may be, for example, a growth factor, a drug, a bulking agent or a biomaterial. In one embodiment, the therapeutic agent is a pro-fibrotic growth factor. The pro-fibrotic growth factor produces scarring in the valve annulus to improve cardiac valve function. In another embodiment, the therapeutic agent is a bulking agent such as collagen. Collagen injected into the valve annulus adds mass to the tissue surrounding the valve. The increased mass causes the valve leaflets to be drawn closer together reducing or eliminating valve leakage. In other embodiments the bulking agent may be, for example, a solidifying liquid or gel or a bead suspension.

FIG. 1 shows an injection catheter in the deflated condition. The injection catheter 20 comprises a catheter 22 with an injection assembly 24 disposed on the distal end. The injection assembly 24 has lobes 26 and a neck 28 between the lobes. Injection ports 30 in the neck 28 provide openings for injectors. The catheter 22 includes lumens to inflate the lobes 26 and deploy the injectors.

Inflatable portions of the injection assembly 24 can be made of a material such as polyethylene, polyethylene terephthalate (PET), nylon, PEBAX® polyether-block amide co-polymers, or the like.

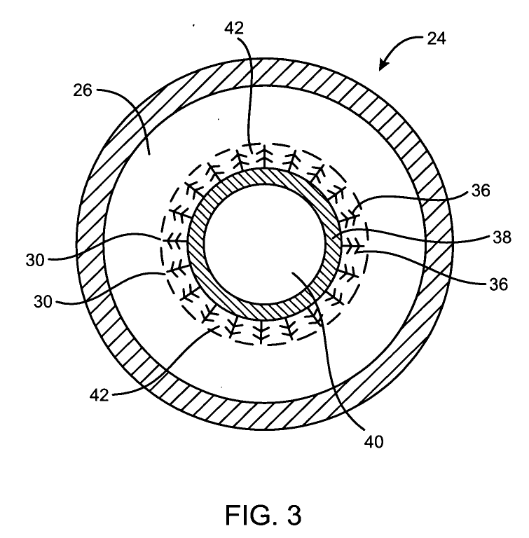

FIG. 2, in which like elements share like reference numbers with FIG. 1, shows an injection catheter in the inflated condition and disposed within a cardiac valve annulus. The inflated lobes 26 center the neck 28 on the cardiac valve annulus 32, so that the injection ports 30 are located at the valve annulus 32. This allows the injectors to be deployed into the valve annulus 32. In one embodiment, the catheter 22 divides into catheter supports 34 to help stabilize the inflated injection assembly 24 within the valve annulus 32. The catheter supports 34 can be connected to the inflatable lobes 26 and separate from each other as the lobes 26 infate. In another embodiment the catheter 22 can be a single, undivided shaft.

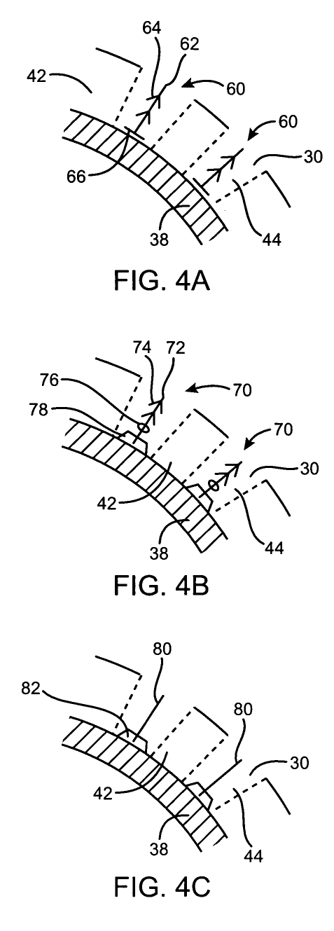

FIG. 3 shows a cross section at A-A of FIG. 2 of the injection catheter in the inflated condition. Injectors 36 are disposed in the injector space 42 behind the ports 30 outward of the inner balloon 38. The inner balloon 38 is disposed in the neck. Inflating the inner balloon 38, once the injection assembly 24 is in place within the valve annulus 32, pushes the injectors 36 into the cardiac valve annulus. A lumen 40 permits blood flow through the injection assembly 24 during the valve modification procedure. In one embodiment, a check valve can be disposed in the lumen 40 to provide valve function during the valve modification procedure.

The injectors 36 can be injection barbs or injection needles. In one embodiment, the injectors 36 can be injection barbs including a therapeutic agent. The injection barbs remain in the valve annulus when the inner balloon 38 is deflated. In another embodiment, the injectors 36 can be injection needles dispensing a therapeutic agent. The therapeutic agent passes through the catheter and the injection needles into the valve annulus. The injection needles retract when the inner balloon 38 is deflated.

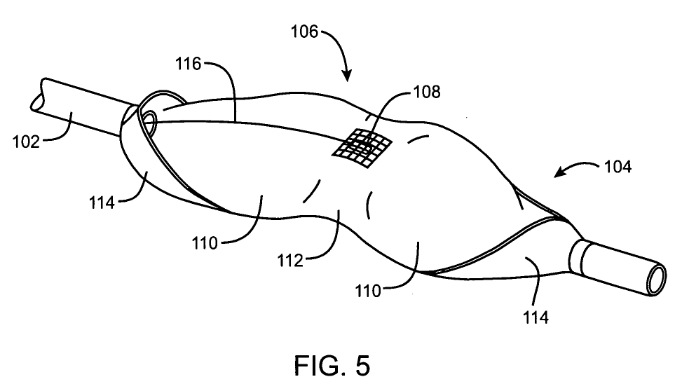

FIGS. 4A-C show detail of several embodiments of the injectors in the injection assembly. The injectors include a therapeutic agent for delivery into the cardiac valve annulus.

FIG. 4A shows a free standing injection barb 60 mounted in an injector bay 44 of the injection assembly. In this embodiment, the injection barb 60 is made of a biodegradable material including a therapeutic agent. The injection barb 60 is delivered into the valve annulus and remains there until the injection barb 60 is absorbed.

The injection barb 60 is disposed in injector bay 44 of the injector space 42 behind port 30. The injection barb 60 comprises a pointed shaft 62, barbs 64, and a foot 66. The foot 66 of the pointed shaft 62 is disposed against the inner balloon 38, so that the inner balloon 38 can push the injection barb 60 from the injector bay 44 into the valve annulus when the inner balloon 38 is inflated. The barbs 64 maintain the injection barb 60 in the valve annulus on implantation. In one embodiment, the port 30 can be covered with a membrane to hold and protect the injection barb 60 as the injection assembly is advanced to the valve annulus. In another embodiment, the injector bay 44 can be filled with a dissolvable viscous material, such as a glutinous or gelatinous material, to enclose, hold, and protect the injection barb 60.

The injection barb 60 can be made of a biodegradable material able to include a therapeutic agent, such as bioabsorbable polymers, including but not limited to polydioxanone, polyglycolic acid (PGA), polylactide (PLA), PGANPLA copolymers, polycaprolactone, poly-b-hydroxybutyrate (PHB), combinations thereof, and the like. In one embodiment, the therapeutic agent can be any therapeutic agent able to produce scarring in the valve annulus, such as pro-fibrotic growth factor, including but not limited to transforming growth factor-β (TGF-β), connective tissue growth factor (CTGF), platelet derived growth factor (PDGF), insulin-like growth factor (IGF), and the like. The pro-fibrotic growth factors induce inflammation in the valve annulus, which heals to create scar tissue. The scar tissue improves the cardiac valve function by changing the flexibility and geometry of the valve annulus. The combination of the biodegradable material and the therapeutic agent can be tuned so that the therapeutic agent is released at a desired rate.

FIG. 4B shows an attached injection barb 70 mounted in an injector bay 44 of the injection assembly. In this embodiment, the injection barb 70 is made of a biodegradable material including a therapeutic agent. The injection barb 70 is delivered into the valve annulus and remains there until the injection barb 70 is absorbed.

The injection barb 70 is disposed in injector bay 44 of the injector space 42 behind port 30. The injection barb 70 comprises a pointed shaft 72, barbs 74, a fracture point 76, and an injector attachment 78. The injector attachment 78 of the pointed shaft 72 is attached to the inner balloon 38, n that the inner balloon 38 can push the injection barb 70 from the injector bay 44 into the valve annulus when the inner balloon 38 is inflated. The barbs 74 maintain the injection barb 70 in the valve annulus, so that the injection barb 70 breaks at the fracture point 76 and leaves the barbs 74 and most of the pointed shaft 72 in the valve annulus as the inner balloon 38 is deflated. The fracture point 76 can be a stress concentrator, such as a notch, or can be a weaker material than the rest of the pointed shaft 72. The injection barb 70 can be made of a biodegradable material including a therapeutic agent as described for the injection barb of FIG. 4A.

Referring to FIG. 4B, the injection barb 70 can be unprotected or protected in the injector bay 44. In one embodiment, the port 30 can be uncovered and the injector bay 44 unfilled. In another embodiment, the port 30 can be covered with a membrane to hold and protect the injection barb 70 as the injection assembly is advanced to the valve annulus. In another embodiment, the injector bay 44 can be filled with a dissolvable gel-like material to hold and protect the injection barb 70.

FIG. 4C shows an attached injection needle 80 mounted in an injector bay 44 of the injection assembly. In this embodiment, the injection needle 80 delivers a therapeutic agent into the valve annulus and is then retracted and removed.

The injection needle 80 is disposed in injector bay 44 of the injector space 42 behind port 30. The injection needle 80 includes a lumen (not shown) and is attached to the inner balloon 38 with an injector attachment 82. The injector attachment 82 can be glue or a mechanical attachment, such as a rivet. The injector attachment 82 pushes the tip of the injection needle 80 from the injector bay 44 into the valve annulus when the inner balloon 38 is inflated. The tip of the injection needle 80 retracts when the inner balloon 38 is deflated. The injection needle 80 can be made of a sharpenable material, such as stainless steel, nitinol, or cobalt based alloys, such as MP35N or L605.

The lumen of the injection needle 80 communicates through the catheter to a therapeutic agent supply outside the patient. The therapeutic agent is injected at the cardiac valve annulus through the injection needle 80. In one embodiment, the therapeutic agent can be any therapeutic agent able to produce scarring in the valve annulus, such as pro-fibrotic growth factor, including but not limited to transforming growth factor-β (TGF-β), connective tissue growth factor (CTGF), platelet derived growth factor (PDGF), insulin-like growth factor (IGF), and the like. The therapeutic agent can be included in microspheres for timed and coordinated release of one or more therapeutic agents. The pro-fibrotic growth factors induce inflammation in the valve annulus, which heals to create scar tissue. The scar tissue improves the cardiac valve function by changing the flexibility and geometry of the valve annulus.

In another embodiment, the therapeutic agent is a bulking agent. The bulking agent may be any therapeutic agent that adds mass to the tissue surrounding the valve when the agent is injected into or adjacent the valve annulus. For example, the bulking agent may be collagen, a bead suspension, a solidifying liquid or gel or an inflammatory agent.

Those skilled in the art will appreciate that many types of injection catheters can be used to deliver a therapeutic agent, such as pro-fibrotic growth factor, to the valve annulus. The injection catheter can have multiple needles injecting at multiple sites in the valve annulus at once, or can have a single needle and the injection catheter can be rotated to inject at multiple sites. In one embodiment, the injection catheter includes a plurality of needles positioned to inject a therapeutic agent into the annulus adjacent the anterior leaflet of the mitral valve. In this embodiment, the therapeutic agent is a bulking agent such as collagen or a beau suspension. An exemplary injection catheter with multiple needles is described in U.S. Pat. No. 6,485,489 to Teirstein et al., incorporated herein by reference. The injection catheter can be steerable to precisely locate the injection assembly at the valve annulus. Examples of such catheters include the MyoCath™ catheter from Bioheart, Inc., the MyoStar catheter from Johnson & Johnson, Inc., and the Stiletto catheter from Boston Scientific, Inc.

The location of the injection catheter relative to the valve annulus can be determined using an imaging or navigation system. In one embodiment, the distal tip of the injection catheter can have a radiopaque marker and fluoroscopy can be used to locate the distal tip. In another embodiment, a non-fluoroscopic navigation system, such as the Localisa® intracardiac navigation system from Medtronic, Inc., of Minneapolis, Minn., can be used to locate the distal tip.

The Localisa® intracardiac navigation system uses three skin electrode pairs, positioned in x,y,z directions around the heart to track catheters. In yet another embodiment, fluoroscopy can be used in conjunction with a non-fluoroscopic navigation system to locate the distal tip.

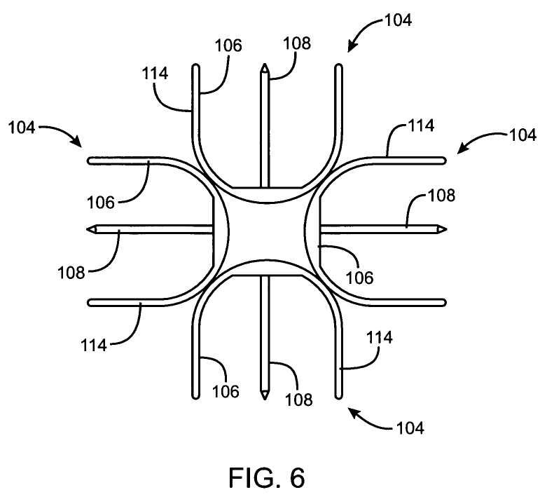

FIGS. 5 & 6 show another injection catheter for cardiac valve modification. The injection catheter uses an injection assembly having at least one balloon with a needle disposed on the balloon. The balloon is inflated to deploy the needle to inject a therapeutic agent, such as pro-fibrotic growth factor or a bulking agent. The basic operation of an injection assembly of the present embodiment is described in U.S. Publication No. 2003/0055400 A1 and U.S. Pat. No. 6,547,803 to Seward, et al., incorporated herein by reference.

FIG. 5 shows an injection catheter in the inflated condition. The injection catheter 100 comprises a catheter 102 with an injection assembly 104 disposed on the distal end. The injection assembly 104 comprises a balloon 106 carrying a needle 108 and an actuator body 114, the balloon 106 being furlable to fit within the actuator body 114 and withdraw the needle 108 in the deflated condition. The balloon 106 comprises lobes 110 and a neck 112 located between the lobes 110. The needle 108 is attached at the neck 112, so that the lobes 110 locate the needle 108 at the cardiac valve annulus when the balloon 106 is inflated. The neck 112 can be created on the balloon 106 by the method of manufacture or varying the materials at the neck 112. A therapeutic agent, such as pro-fibrotic growth factor, can be injected through the needle 108 of the injector by means of the fluid line 116 communicating outside the patient through the catheter 102. The pro-fibrotic growth factor creates scar tissue to improve cardiac valve function by changing the flexibility and geometry of the valve annulus.

FIG. 6, in which like elements share like reference numbers with FIG. 5, shows a cross section of a multi-needle injection catheter in the deflated condition. In this embodiment, several injection assemblies 104 are connected with the needles 108 pointing out from the center to form a multi-needle injection catheter. FIG. 6 shows the cross section at the needles 108, which are attached to the necks of the balloons 106. When deployed, the balloons 106 extend the needles 108 from the actuator bodies 114. In the inflated condition, the combined necks of the several injection assemblies 104 are smaller than the combined lobes, so that the necks locate the needles 108 at the smaller valve annulus. The actuator bodies 114 can be attached with glue or mechanical fasteners, or can be manufactured as an integral unit.

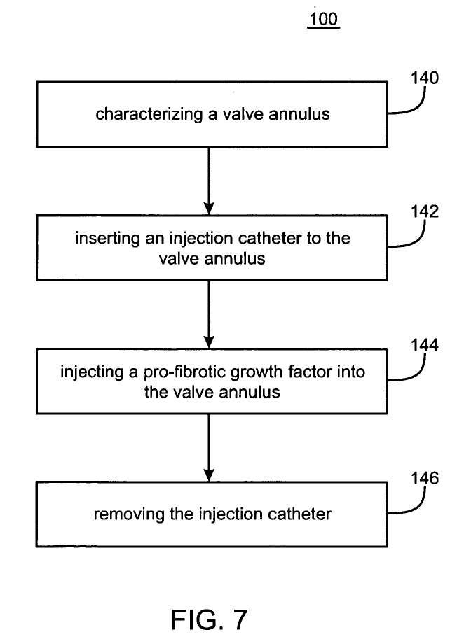

FIG. 7 shows a flowchart for a method 100 of cardiac valve modification. A valve annulus is characterized (Block 140). An injection catheter is inserted to the valve annulus (Block 142) and a pro-fibrotic growth factor injected into the valve annulus with the injection catheter (Block 144). The injection catheter is removed (Block 146).

Characterizing the valve annulus (Block 140) can comprise identifying target sites in the valve annulus where injection of a pro-fibrotic growth factor would improve valve function. The pro-fibrotic growth factor changes the flexibility and geometry of the valve annulus. Characterizing the valve annulus (Block 140) can be performed by echocardiography, using echocardiography to identify valve leakage. In other embodiments, magnetic resonance imaging (MRI) or ultrafast computed tomography (CT) can be used to characterize the valve annulus.

While the injection catheter is transvascularly inserted to the valve annulus (Block 142), the injection catheter can be located by an imaging or navigation system, such as fluoroscopy or a Localisa® non-fluoroscopic intracardiac navigation system from Medtronic, Inc. The injection catheter can be single needle or multi-needle, and can be steerable. Inserting the injection catheter to the valve annulus (Block 142) can also comprise locating an injection assembly at the valve annulus, or deploying an injection assembly at the valve annulus, such as inflating an injection assembly.

Injecting pro-fibrotic growth factor into the valve annulus with the injection catheter (Block 144) can comprise injecting pro-fibrotic growth factor at target sites identified while characterizing the valve annulus. The pro-fibrotic growth factor can be a pro-fibrotic growth factor such as transforming growth factory (TGF-β), connective tissue growth factor (CTGF), platelet derived growth factor (PDGF), insulin-like growth factor (IGF), and the like. The pro-fibrotic growth factor can be included in microspheres for timed and coordinated release of one or more therapeutic agents.

In one embodiment, the method of cardiac valve modification can further comprise performing sensitivity testing to determine the proper pro-fibrotic growth factor and the sensitivity to particular pro-fibrotic growth factors for a particular patient. Different patients react differently, so sensitivity testing assures that the dose of a particular pro-fibrotic growth factor will achieve the desired amount of modification in the valve annulus. The sensitivity testing can be performed by injecting the patient subcutaneously with varied pro-fibrotic growth factors in varied strengths and measuring the inflammation and scarring reaction.

In another embodiment, the method of cardiac valve modification can further comprise performing a series of cardiac valve modification procedures, modifying the valve annulus in stages until the valve function is satisfactory. The inflammation and scarring process typically takes days to weeks after injecting pro-fibrotic growth factor, so the procedures can be performed weeks to months apart. The valve function can be checked after each procedure, using echocardiography or another similar method. Pro-fibrotic growth factor is injected into the valve annulus with each procedure if the valve function can be improved. The series approach assures that too great a modification is not applied in any one operation.

In another embodiment, the method of cardiac valve modification can further comprise checking the valve function after the cardiac valve modification procedure and repeating the cardiac valve modification procedure until valve function is satisfactory. The inflammation and scarring process typically takes days to weeks, so the checking of the results achieved is performed after that time. Checking the valve function can comprise checking the valve function using echocardiography. In other embodiments, magnetic resonance imaging (MRI) or ultrafast computed tomography (CT) can be used to check the valve function. If the further improvement in valve function is possible, the cardiac valve modification procedure can be repeated.

One method to monitor the progress of the inflammation and scarring process is to monitor C-reactive protein (CRP). CRP is an acute phase reactant released by the body in response to acute injury, infection, or other inflammatory stimuli. The inflammation in the valve annulus will release CRP. A decline in CRP following the cardiac valve modification procedure indicates the scarring is substantially complete. Monitoring the CRP will only be effective if no other acute injury, infection, or other inflammatory stimuli are present in the patient.

In another embodiment, the method of cardiac valve modification can further comprise modifying the scar tissue formed in the valve annulus by the cardiac valve modification procedure to further improve valve function. The scar tissue can be injected with a therapeutic agent, such as gluteraldehyde or other similar agents, to contract the scar tissue and further tighten the valve annulus.

The therapeutic agent can be injected with an injection catheter as described herein for the injection of pro-fibrotic growth factors. Exposure to ultraviolet radiation can also be used to contract the scar tissue, by applying the ultraviolet radiation directly to the scar tissue.

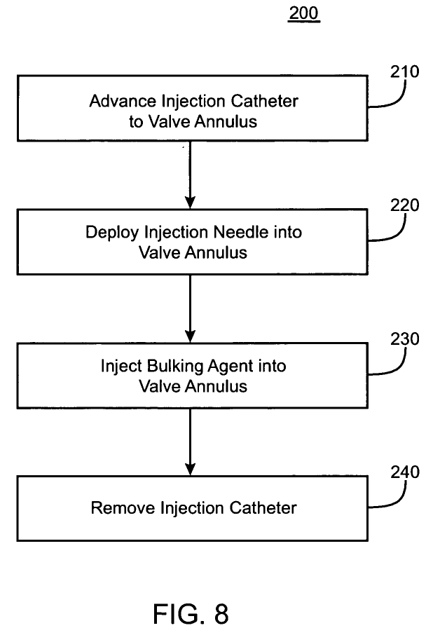

FIG. 8 shows a flowchart for a method 200 of cardiac valve modification. Method 200 may utilize an injection catheter as described above in relation to FIGS. 1-6. Method 200 uses a bulking agent to modify the valve annulus. A distal end of an injection catheter is advanced via a transvascular (percutaneous) pathway to a position adjacent to the valve annulus (Block 210). Once the distal end of the injection catheter is properly positioned, the injection assembly disposed on the distal end of the injection catheter is actuated to deploy the injection needle or needles (Block 220).

The bulking agent is then injected into the valve annulus (Block 230). The bulking agent may be any bulking agent or combination of bulking agents known to those with skill in the art. In one embodiment, the bulking agent is collagen. In another embodiment, the bulking agent is a suspension of beads. The injection catheter is removed after the bulking agent has been injected (Block 240).

The bulking agent will modify the size and/or shape of the valve annulus by increasing the mass of the tissue surrounding the valve. The increase in tissue mass results in bringing the valve leaflets closer together, reducing or eliminating valve leakage.

In one embodiment, method 200 includes an injection assembly for treating a portion of a mitral valve annulus. In this embodiment, the injection assembly includes a plurality of needles positioned so that, when deployed, the needles are inserted only into the portion of the valve annulus adjacent the anterior leaflet of the valve. The bulking agent is then injected into the valve annulus only in the area adjacent the anterior leaflet. Injecting the bulking agent in this portion of the annulus is advantageous. This area of the mitral valve annulus is more fibrotic and dense as compared to the posterior side of the valve annulus providing a more stable region for pushing the valve leaflet into place.

It is important to note that FIGS. 1-8 illustrate specific applications and embodiments of the present invention, and is not intended to limit the scope of the present disclosure or claims to that which is presented therein. Upon reading the specification and reviewing the drawings hereof, it will become immediately obvious to those skilled in the art that myriad other embodiments of the present invention are possible, and that such embodiments are contemplated and fall within the scope of the presently claimed invention.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A device for cardiac valve modification comprising:
a catheter;
an injection assembly disposed on the catheter, the injection assembly having a first lobe, a second lobe, and a neck disposed between the first lobe and the second lobe; and
at least one injector operably disposed at the neck.

2. The device of claim 1 wherein the first lobe and the second lobe are inflatable.

3. The device of claim 1 wherein the injector is selected from the group consisting of an injection barb and an injection needle.

4. The device of claim 1 wherein the neck includes an inner balloon and at least one port, the injector being disposed behind the port and the inner balloon being disposed behind the injector.

5. The device of claim 4 wherein the port is closed with a membrane.

6. The device of claim 4 wherein the injector is enclosed in a viscous material.

7. The device of claim 4 wherein the injector comprises a pointed shaft, at least one barb disposed on the pointed shaft, an injector attachment, and a fracture point disposed between the barb and the injector attachment, the injector attachment being attached to the inner balloon.

8. The device of claim 4 wherein the injector comprises an injection needle attached to the inner balloon.

9. The device of claim 8 wherein the injection needle includes a lumen for delivery of a therapeutic agent, the therapeutic agent selected from the group consisting of pro-fibrotic growth factor, transforming growth factor-β (TGF-β), connective tissue growth factor (CTGF), platelet derived growth factor (PDGF), insulin-like growth factor (IGF), and the like.

10. The device of claim 9 wherein the therapeutic agent is included in microspheres.

11. The device of claim 8 wherein the injection needle includes a lumen for delivery of a bulking agent.

12. The device of claim 11 wherein the bulking agent is collagen.

13 The device of claim 1 wherein the injector comprises a biodegradable material and a therapeutic agent.

14. The device of claim 13 wherein the biodegradable material is selected from the group consisting of bioabsorbable polymers, polydioxanone, polyglycolic acid (PGA), polylactide (PLA), PGA/PLA copolymers, polycaprolactone, poly-b-hydroxybutyrate (PHB), combinations thereof, and the like.

15. The device of claim 13 wherein the therapeutic agent is selected from the group consisting of pro-fibrotic growth factor, transforming growth factor-β (TGF-β), connective tissue growth factor (CTGF), platelet derived growth factor (PDGF), insulin-like growth factor (IGF), and the like.

16. The device of claim 1 wherein the injector comprises a pointed shaft, and at least one barb disposed on the pointed shaft.

17. The device of claim 16 wherein the injector has a pointed end, and the injector further comprises a foot disposed on the pointed shaft opposite the pointed end.

18. The device of claim 1 wherein the injection assembly includes a lumen.

19. The device of claim 18 further comprising a check valve disposed in the lumen.

20. The device of claim 1 wherein the injection assembly further comprises a balloon and an actuator body, the balloon including the first lobe, the second lobe, and the neck disposed between the first lobe and the second lobe; the injector comprising a needle; and the balloon being furled within the actuator body when the balloon is deflated.

21. A method of cardiac valve modification comprising:
inserting an injection catheter to the valve annulus;
injecting a therapeutic agent into the valve annulus with the injection catheter; and
removing the injection catheter.

22. The method of claim 21 further comprising:
characterizing a valve annulus;

23. The method of claim 22 wherein characterizing a valve annulus comprises characterizing a valve annulus by a method selected form the group consisting of echocardiography, magnetic resonance imaging, and ultrafast computed tomography.

24. The method of claim 21 wherein injecting the therapeutic agent into the valve annulus with the injection catheter further comprises injecting pro-fibrotic growth factor at a target site identified from characterizing the valve annulus.

25. The method of claim 21 wherein inserting an injection catheter to the valve annulus further comprises locating an injection assembly at the valve annulus.

26. The method of claim 21 wherein inserting an injection catheter to the valve annulus further comprises deploying an injection assembly at the valve annulus.

27. The method of claim 21 wherein inserting an injection catheter to the valve annulus further comprises steering the injection catheter to the valve annulus.

28. The method of claim 21 wherein inserting an injection catheter to the valve annulus further comprises tracking the injection catheter using a system selected from the group consisting of a fluoroscopic system, a non-fluoroscopic navigation system, and a combination thereof.

29. The method of claim 21 wherein the injection catheter is selected from the group consisting of single needle catheters and multi-needle catheters.

30. The method of claim 24 further comprising testing a patient for sensitivity to the pro-fibrotic growth factor.

31. The method of claim 24 wherein injecting pro-fibrotic growth factor into the valve annulus generates scar tissue in the valve annulus, and further comprising checking valve function after the scar tissue forms.

32. The method of claim 31 further comprising injecting pro-fibrotic growth factor into the valve annulus if the valve function can be improved.

33. The method of claim 21 further comprising monitoring inflammation of the valve annulus.

34. The method of claim 33 wherein monitoring inflammation of the valve annulus comprises monitoring C-reactive protein (CRP).

35. The method of claim 24 wherein injecting pro-fibrotic growth factor into the valve annulus generates scar tissue in the valve annulus, and further comprising modifying the scar tissue.

36. The method of claim 35 wherein modifying the scar tissue comprises modifying the scar tissue by a method selected from the group consisting of injecting the scar tissue with gluteraldehyde, and exposing the scar tissue to ultraviolet light.

37. The method of claim 21 wherein injecting a therapeutic agent into the valve annulus comprises injecting a bulking agent.

38. The method of claim 29 wherein the multi-needle injection catheter comprises a plurality of needles positioned for deployment into a portion of the valve annulus.

39. The method of claim 38 where in the needles are positioned for deployment into the portion of the valve annulus adjacent an anterior leaflet of a mitral valve.

40. A system for cardiac valve modification comprising:
means for modifying a valve annulus;
means for injecting the modifying means into the valve annulus; and
means for locating the injecting means at the valve annulus.

41. The system of claim 40 wherein the modifying means is a scarring means selected from the group consisting of pro-fibrotic growth factor, transforming growth factor-β (TGF-β), connective tissue growth factor (CTGF), platelet derived growth factor (PDGF), insulin-like growth factor (IGF), and the like.

42. The system of claim 40 further comprising means for deploying the injecting means

43. The system of claim 40 further comprising means for tracking the injecting means.

44. The system of claim 41 further comprising means for testing patient sensitivity to the scarring means.

45. The system of claim 40 further comprising means for monitoring inflammation of the valve annulus.

46. The system of claim 41 wherein the scarring means produces scar tissue in the valve annulus and further comprising means for modifying the scar tissue.
